(19) United States
(12) Patent Application Publication
(10) Pub. No.: US 2025/0205205 A1
(43) Pub. Date: Jun. 26, 2025

(54) METHODS AND COMPOSITIONS FOR PREVENTING AND TREATING FIBROSIS AND IMPROVING FUNCTIONAL RECOVERY AFTER INJURY OR WITH AGING

(71) Applicant: East Carolina University, Greenville, NC (US)

(72) Inventors: Stefan Clemens, Greenville, NC (US); Kori Brewer, Greenville, NC (US); Mandee Kate Schaub, Richardson, TX (US)

(21) Appl. No.: 18/844,290

(22) PCT Filed: Apr. 12, 2023
(86) PCT No.: PCT/US2023/065656

§ 371 Date: Sep. 5, 2024

Related U.S. Application Data
(60) Provisional application No. 63/330,071, filed on Apr. 12, 2022.
(60) Provisional application No. 63/383,382, filed on Nov. 11, 2022.

(51) Int. Cl. A61K 31/428; A61K 31/485; A61K 45/06

(52) U.S. Cl. CPC A61K 31/428; A61K 31/485; A61K 45/06

(57) ABSTRACT

The present inventive concept provides methods for reducing, treating and/or managing inflammation across a wide range of states associated with injury, disease, and/or aging; reducing or inhibiting disease progression and fibrosis, and for improving functional recovery including improvements and/or maintenance in nerve function, recovery, and damage wherein diseases and/or disorders associated with injury to nerve signaling can be completely or partially restored or stabilized. The inventive concept further provides compositions and kits for use in the methods described herein.

### CROSS-REFERENCE TO RELATED APPLICATIONS

The present application is a 35 USC § 371 national phase application of International Application No. PCT/US2023/065656, filed Apr. 12, 2023, which claims the benefit of U.S. Provisional Patent Application No. 63/330,071, filed Apr. 12, 2022, and U.S. Provisional Patent Application No. 63/383,382, filed Nov. 11, 2022, the disclosures of each of which are hereby incorporated herein by reference.

### RESERVATION OF COPYRIGHT

A portion of the disclosure of this patent document contains material, which is subject to copyright protection. The copyright owner, East Carolina University, Greenville, North Carolina, a constituent institution of the University of North Carolina, has no objection to the reproduction by anyone of the patent document or the patent disclosure, as it appears in U.S. Patent and Trademark Office patent files or records, but otherwise reserves all copyright rights whatsoever.

### FIELD

The present inventive concept is directed to methods and compositions for reducing, treating and/or managing states associated with injury, disease and/or aging, for example, but not limited to, inflammation, reducing or inhibiting disease progression, reducing or inhibiting inflammation, and/or for improving functional recovery, including methods of reducing, treating and/or managing fibrosis associated with injury, disease and/or aging, for example, but not limited to, inflammation, reducing or inhibiting disease progression, reducing or inhibiting inflammation, and/or for improving functional recovery, including improvements and/or maintenance in nerve function, recovery, and functional recovery from damage resulting from diseases and/or disorders associated with injury to nerve signaling can be completely or partially restored or stabilized.

### BACKGROUND

Fibrosis is the result of a gradual accumulation of fibrous connective tissue in response to injury and/or damage due to injury or disease. Fibrosis is a major pathological feature and is associated with of and is associated with many diseases including, neurodegenerative disease, autoimmune disease, cancer, cardiovascular disease and tissue/organ injury as well as specific triggers such as inherited genetic disorders; persistent infections; recurrent exposure to toxins, irritants or smoke; inflammation, including chronic inflammation, for example, chronic autoimmune inflammation; minor human leukocyte antigen mismatches in transplants; myocardial infarction; high serum cholesterol; obesity; and poorly controlled diabetes and hypertension.

Fibrosis, and fibrosis associated with inflammation, is also associated with the normal aging process, contributing to the medical comorbidities listed above. Targeting fibrosis development and reducing the excess deposition of extracellular matrix (ECM) has been examined by numerous research laboratories worldwide for potential clinical impact. A key observation of fibrosis is modulation of membrane and action potentials in response to stimuli and injury. Interestingly, pharmacological approaches that raise cyclic AMP (cAMP) have been postulated to block or reverse tissue fibrosis.

Fibrosis is believed to be the end result of inflammatory reactions, such as chronic inflammatory reactions induced by a variety of stimuli including persistent infections, autoimmune reactions, allergic responses, chemical insults, radiation, and tissue injury (Wynn (2008) J. Pathol. 214(2), 199-210). In addition, there is a growing appreciation that peripheral immune cell infiltration into the CNS parenchyma can have detrimental consequences in autoimmune disease, as well as in disorders such as ischemic stroke, traumatic injury, and neurodegenerative disease. An important and well-recognized consequence of such inflammation in other organs throughout the body is the proliferation of fibroblast-lineage stromal cells, which results in scar formation and fibrosis due to accumulation of extracellular matrix proteins such as collagens (Sofroniew (2021) Nat. Neurosci. 24, 157-159). Furthermore, it has been noted that senescence is an irreversible replicative-arrest state of cells, leading to changes in gene expression and phenotype that alter the function of neighboring cells. Senescent cells release pro-inflammatory signals (e.g., Interleukins (IL), IL-1α, IL-1β, IL-6, and IL-8) that are referred to as the “senescence-associated secretory phenotype” (SASP). Accumulation of senescent cells during aging promotes chronic inflammation and tissue dysfunction and is an essential contributor to the progression of age-associated diseases, such as AD and atherosclerosis (Finger et al. (2022) Mol. Psychiatry 27, 803-818). Moreover, in traumatic brain injury (TBI), inflammatory mediators have been observed to simultaneously enhance immune cell recruitment to eliminate senescent cells and perpetuate inflammation. Cells exhibiting the SASP also release matrix metalloproteinases (MMP), including MMP-3, MMP-9, MMP-12 and serpins, such as plasminogen activator inhibitor-1 (PAI-1) that modify the extracellular matrix and induce both tissue fibrosis and destruction. Fibrosis and glial scarring have been observed to persist for years after TBI and contribute to chronic neurodegeneration (Lu et al. (2023) Neurobiology of Disease 180, 106090).

The present inventive concept overcomes previous shortcomings in the art by providing methods and compositions for treating and/or managing states associated with injury, disease, and/or aging, reducing or inhibiting disease progression and inflammation, and for improving functional recovery, including methods of reducing, treating and/or managing fibrosis across a wide range of disease and injury states, for example, treating and/or managing inflammation driven by injury, disease, and/or aging, treating and/or managing inflammation that results in and/or is associated with fibrosis, and treating and/or managing fibrosis that is associated with inflammation.

### SUMMARY

Aspects of the present inventive concept provide methods for reducing, treating and/or managing inflammation across a wide range of states associated with injury, disease, and/or aging. The inventive concept further provides methods for reducing and/or inhibiting disease progression and/or inflammation, for example, reducing and/or inhibiting inflammation, which in some aspects, may be achieved by reducing fibrosis associated with inflammation. These aspects include, but are not limited to, improvements and/or maintenance in nerve function, recovery, and damage wherein diseases and/or disorders associated with injury to nerve signaling can be completely or partially restored or stabilized.

The present inventive concept may be achieved by administering to the subject an effective amount of an opioid and a dopamine type 3 receptor (D3R) agonist or an effective amount of an opioid and a dopamine type 1 receptor (D1R) antagonist.

Accordingly, in an aspect of the inventive concept, provided is a method of reducing, treating, and/or managing fibrosis associated with disease or aging, reducing or inhibiting disease progression and inflammation, and/or for improving functional recovery including administering to the subject an effective amount of an opioid and a dopamine type 3 receptor (D3R) agonist or an effective amount of an opioid and a dopamine type 1 receptor (D1R) antagonist.

In another aspect, provided is a composition including: (a) at least one opioid; (b) at least one dopamine type 3 receptor agonist or a dopamine type 1 receptor antagonist; (c) an additional therapeutic agent; and (d) a pharmaceutically acceptable carrier, excipient or diluent.

In another aspect, provided is a kit for reducing, treating and/or managing fibrosis associated with disease or aging, reducing or inhibiting disease progression and inflammation, and/or for improving functional recovery comprising at least one opioid and at least one dopamine type 3 receptor agonist or a dopamine type 1 receptor (D1R) antagonist.

In another aspect, provided is a method of reducing, treating and/or managing a neurodegenerative disease in a subject, the method including: taking a baseline compound action potential (CAP) measurement; treating the subject with at least one opioid and at least one dopamine type 3 receptor (D3R) agonist or a dopamine type 1 receptor (D1R) antagonist; taking an additional baseline compound action potential (CAP) measurement after treating the subject with the at least one opioid and at least one D3R agonist or the D1R antagonist; and assessing whether the CAP (amplitude, area, conduction velocity, and latency) is stabilized or improves over time.

In another aspect, provided is a method of reducing, treating, and/or managing inflammation resulting from injury, disease, and/or aging in a subject, the method including administering to the subject an effective amount of an opioid, and a dopamine type 3 receptor (D3R) agonist or an effective amount of an opioid and a dopamine type 1 receptor (D1R) antagonist.

In another aspect, provided is a kit for reducing, treating and/or managing inflammation associated with disease or aging, reducing or inhibiting disease progression and/or fibrosis associated with the inflammation, and for improving functional recovery including at least one opioid and at least one dopamine type 3 receptor agonist or a dopamine type 1 receptor (D1R) antagonist.

### BRIEF DESCRIPTION OF THE DRAWINGS

FIG. 1 depicts proof-of-concept in vitro data showing the effect of sciatic nerve injury and the responses of different long-term treatment on fibrosis. Under control conditions, the sciatic nerve expresses only low levels of fibrosis (black bar). After injury, and after long-term treatment with saline only, the fibrosis level around the site of the injury is strongly increased (white bar). Long-term treatment with a pramipexole (PPX) and morphine combination (0.5 mg/kg and 1 mg/kg each) was associated with a prominent decrease of the fibrosis signal (gray bar).

FIG. 2 depicts the in vitro effects of different long-term treatments on sciatic nerve function, as assessed by the response profiles of these nerves to electrical stimulation. Panel A shows on the left the compound action potentials (CAPs, s. arrow) of a representative animal that had undergone sciatic nerve injury and was exposed to daily treatments of 100 μl saline (the drug carrier for the experiments in B-D). In the same animal, on the injured side, stimulation of the nerve failed to elicit a similarly strong CAP as on the injured contralateral side (labeled by ‘X’). Panel B shows on the left the CAPs of a representative animal that had undergone sciatic nerve injury and was exposed to daily treatments of 100 μl PPX (0.5 mg/kg, dissolved in saline). In the same animal, on the injured side, stimulation of the nerve failed to elicit a similarly strong CAP as on the injured contralateral side (labeled by ‘X’). Panel C shows on the left the CAPs of a representative animal that had undergone sciatic nerve injury and was exposed to daily treatments of 100 μl morphine (1 mg/kg, dissolved in saline). In the same animal, on the injured side, stimulation of the nerve failed to elicit a similarly strong CAP as on the injured contralateral side (labeled by ‘X’). Panel D shows on the left the CAPs of a representative animal that had undergone sciatic nerve injury and was exposed to daily treatments of 100 μl PPX: morphine (0.5 mg/kg and 1 mg/kg, respectively, dissolved in saline). In the same animal, on the injured side, stimulation of the nerve was able to produce a CAP, albeit less strong than on the injured contralateral side (s. arrow).

FIG. 3 depicts the in vivo effects of different long-term treatments on pain withdrawal reflex latencies. Panel A shows the effects of saline treatment on reflex latencies after unilateral sciatic nerve injury. Hollow squares show the reflex data of the contralateral (un-injured) side of the animal, which stay stable over time. Filled circles show the reflex data of the injured side of the animal, and they show that saline treatment did not lead to a recovery of withdrawal latencies to pre-injury data (days −3 to −1). Panel B shows the effects of PPX treatment (0.5 mg/kg) on reflex latencies after unilateral sciatic nerve injury. Hollow squares show the reflex data of the contralateral (un-injured) side of the animal, which stay stable over time. Filled circles show the reflex data of the injured side of the animal. As with saline treatment, treatment with PPX alone did not lead to a recovery of withdrawal latencies to pre-injury data (days −3 to −1). Panel C shows the effects of morphine treatment (1 mg/kg) on reflex latencies after unilateral sciatic nerve injury. Hollow squares show the reflex data of the contralateral (un-injured) side of the animal, which stay stable over time. Filled circles show the reflex data of the injured side of the animal. As with saline or PPX treatment, treatment with morphine alone did not lead to a recovery of withdrawal latencies to pre-injury data (days −3 to −1). Panel D shows the effects of the PPX: morphine treatment (0.5 mg/kg and 1 mg/kg, respectively) on reflex latencies after unilateral sciatic nerve injury. Hollow squares show the reflex data of the contralateral (un-injured) side of the animal, which stay stable over time. Filled circles show the reflex data of the injured side of the animal. Unlike PPX or morphine treatment alone, treatment with the PPX: morphine combination maintained withdrawal latencies of the injured side at the levels of the pre-injury data (days −3 to −1).

### DETAILED DESCRIPTION

The present inventive concept will now be described with reference to the following embodiments. As is apparent by these descriptions, the inventive concept can be embodied in different forms and should not be construed as limited to the embodiments set forth herein. Rather, these embodiments are provided so that this disclosure will be thorough and complete, and will fully convey the scope of the inventive concept to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which the inventive concept belongs. The terminology used in the description of the inventive concept herein is for the purpose of describing particular embodiments only and is not intended to be limiting of the inventive concept.

All publications, patent applications, patents, and other references mentioned herein are incorporated by reference in their entirety.

The present inventive concept is based on the unexpected discovery that a recovery of compound action potentials (CAPs) following a peripheral injury when treating the animals with the combination of an opioid receptor agonist and a D2-like receptor agonist. In contrast, no changes in CAPs were observed after treating injured animals with either an opioid agonist or the dopamine receptor agonist alone. This novel discovery opens the door for a novel treatment for fibrosis across a wide range of disease and injury states, as well as wherein diseases and/or disorders associated with injury to nerve signaling.

Thus, in some embodiments, the present inventive concept provides methods of reducing, treating and/or managing states associated with injury, disease and/or aging, for example, but not limited to, inflammation, reducing or inhibiting disease progression, reducing or inhibiting inflammation, and/or for improving functional recovery, including methods of reducing, treating and/or managing fibrosis associated with injury, disease or aging, reducing or inhibiting disease progression, reducing or inhibiting inflammation, and for improving functional recovery including administering to the subject an effective amount of an opioid and a dopamine type 3 receptor (D3R) agonist and/or an effective amount of an opioid and a dopamine type 1 receptor (D1R) antagonist.

Accordingly, in some embodiments, the present inventive concept provides methods of reducing, treating, and/or managing inflammation associated with injury, disease or aging, for example, reducing or inhibiting disease progression, reducing or inhibiting inflammation, and/or for improving functional recovery, including methods of reducing, treating and/or managing fibrosis associated with inflammation, and fibrosis associated with injury, disease or aging, reducing or inhibiting disease progression, reducing or inhibiting inflammation, and for improving functional recovery including administering to the subject an effective amount of an opioid and a dopamine type 3 receptor (D3R) agonist and/or an effective amount of an opioid and a dopamine type 1 receptor (D1R) antagonist.

In each of the methods of the inventive concept, the effect of the combination treatment, can be quantitated by methods that are well known in the art and as described herein.

In the methods of the inventive concept, the opioid and the D3R agonist and/or the opioid and the D1R antagonist can be administered in the same composition or formulation and/or in separate compositions or formulations. Said separate compositions and/or formulations can be administered simultaneously, concurrently and/or in any order and/or in any interval of minutes, days, weeks, etc. In some embodiments, the D3R agonist or the D1R antagonist may be administered about 1, 2, 3, 4, 5, 10, 15, 20, 25, 30, 35, 40, 45, 50, or 55 minutes or 1, 2, 3, 4, 5, 6, 12, or 18 hours or 1, 2, 3, 4, 5, 6, or 7 or more days before or after administration of the opioid. One or more doses of the D3R agonist or D1R antagonist may be administered before or after one or more doses of the opioid.

Fibrosis is associated with a wide range of disease and injury states, including neurodegenerative disease, autoimmune disease, cancer, cardiovascular disease, tissue/organ injury as well as specific triggers such as inherited genetic disorders; injury repair; repeated injuries; persistent infections; recurrent exposure to toxins, irritants, or smoke; inflammation, for example, chronic inflammation, including chronic autoimmune inflammation; minor human leukocyte antigen mismatches in transplants; myocardial infarction; high serum cholesterol; obesity; diabetes, and hypertension, as well as aging.

Nerve function, recovery, and/or damage includes nerve injury from neuropraxia, axonotmesis, neurotmesis, spinal cord injury, traumatic brain injury, dementia, Alzheimer's disease, Parkinson's disease, motor neuron disease, frontotemporal degenerations, extrapyramidal disorders, spinocerebellar degenerations, principal molecular abnormality, amyloidoses, tauopathies, a-synucleinopathies, TDP-43 proteinopathies, memory disorders, Huntington's disease, Parkinson's disease, Motor neuron disease, Multiple system atrophy, Progressive supranuclear palsy, Aphasia, neuromuscular disorders, Palsy, spinal muscular atropy, lewy body disease, ataxia, sclerosis, Batten disease, Cerebro-Oculo-Facio-Skeletal Syndrome, Corticobasal Degeneration, Kuru, Leigh's Disease, amyotrophy, atrophy, prion disease, encephalopathies, striatonigral degeneration, and genetic neurodegenerative disease. In some embodiments the disease is Alzheimer's disease, Dementia, Parkinson's, Multiple Sclerosis, ALS and Huntington's all could be disease targets.

Improvements and/or treatments for nerve function, recovery, and/or damage include impacts on mobility and balance, abnormal movements, swallowing, bladder and bowel function, blood pressure fluctuation, sleep, breathing, heart function, memory abilities, cognitive abilities, mood, speech, nerve conduction velocities, axon and nerve fiber function, compound action potentials, cell membrane potentials, neuron firing rates, neuron properties, cell membrane potentials, action potential firing rates, action potential thresholds, rheobase, and membrane resistance.

Improvements and/or treatments for nerve function, recovery, and damage may also include treatments for nerve function, recovery, and/or damage resulting from an infection, and in some embodiments, a persistent infection. The infection may be bacterial or viral without limitation, that may result in inflammation and/or fibrosis, e.g., from encephalitis, which may result in cognitive decline and/or dementia.

Aging is accompanied by structural and neurophysiological changes that drive cognitive decline and susceptibility to degenerative disorders in healthy individuals. These changes may include synapse loss and loss of neuronal function. Neurons in the aging brain are vulnerable to alterations in structure, synaptic integrity, and molecular processing at both synaptic and the axonal level, either or all of which impair cognitive function. In addition to the normal synapse loss during natural aging, synapse loss and axonal degradation are early pathological event common to many neurodegenerative conditions. Diseases associated with aging include, but are not limited to atherosclerosis, cardiovascular disease, cancer, arthritis, cataracts, osteoporosis, type 2 diabetes, hypertension, hearing loss, muscle weakness, cell senescence, urologic changes, dementia, mobility disorders, frailty, continence, depression, memory, lung function, genetic aging disease, vision loss, collagen loss and Alzheimer's disease.

Nonlimiting examples of an opioid that can be administered according to a method of the inventive concept include generic opioid drugs (e.g., morphine sulfate, fentanyl, methadone hydrochloride, oxymorphone hydrochloride); brand name opioid drugs (e.g., Abstral (fentanyl), Actiq (fentanyl), Avinza (morphine sulfate extended-release capsules), Butrans (buprenorphine transdermal system), Demerol (meperidine [also known as isonipecaine or pethidine]), Dilaudid (hydromorphone [also known as dihydromorphinone]), Dolophine (methadone hydrochloride tablets), Duragesic (fentanyl transdermal system), Fentora (fentanyl), Hysingla (hydrocodone), Methadose (methadone), Morphabond (morphine), Nucynta ER (tapentadol extended-release oral tablets), Onsolis (fentanyl), Oramorph (morphine), Oxaydo (oxycodone), Roxanol-T (morphine), Sublimaze (fentanyl), Xtampza ER (oxycodone), Zohydro ER (hydrocodone)); and combination formulations of opioid drugs (e.g., Anexsia (hydrocodone containing acetaminophen), Co-Gesic (hydrocodone containing acetaminophen), Embeda (morphine sulfate and naltrexone extended-release capsules), Exalgo (hydromorphone hydrochloride extended-release tablets), Hycet (hydrocodone containing acetaminophen), Hycodan (hydrocodone containing homatropine), Hydromet (hydrocodone containing homatropine), Ibudone (hydrocodone containing ibuprofen), Kadian (morphine sulfate extended-release tablets), Liquicet (hydrocodone containing acetaminophen), Lorcet (hydrocodone containing acetaminophen), Lorcet Plus (hydrocodone containing acetaminophen), Lortab (hydrocodone containing acetaminophen), Maxidone (hydrocodone containing acetaminophen), MS Contin (morphine sulfate controlled-release tablets), Norco (hydrocodone containing acetaminophen), Opana ER (oxymorphone hydrochloride extended-release tablets), OxyContin (oxycodone hydrochloride controlled-release tablets), Oxycet (oxycodone containing acetaminophen), Palladone (hydromorphone hydrochloride extended-release capsules), Percocet (oxycodone containing acetaminophen), Percodan (oxycodone containing aspirin), Reprexain (hydrocodone containing ibuprofen), Rezira (hydrocodone containing pseudoephedrine), Roxicet (oxycodone containing acetaminophen), Targiniq ER (oxycodone containing naloxone), TussiCaps (hydrocodone containing chlorpheniramine), Tussionex (hydrocodone containing chlorpheniramine), Tuzistra XR (codeine containing chlorpheniramine), Tylenol #3 and #4 (codeine containing acetaminophen), Vicodin (hydrocodone containing acetaminophen), Vicodin ES (hydrocodone containing acetaminophen), Vicodin HP (hydrocodone containing acetaminophen), Vicoprofen (hydrocodone containing ibuprofen), Vituz (hydrocodone containing chlorpheniramine), Xartemis XR (oxycodone containing acetaminophen), Xodol (hydrocodone containing acetaminophen), Zolvit (hydrocodone containing acetaminophen), Zutripro (hydrocodone containing chlorpheniramine and pseudoephedrine), Zydone (hydrocodone containing acetaminophen)). The opioids of the inventive concept can be employed in the methods of the inventive concept singly or in any combination and/or ratio.

The present inventive concept is achieved by administering to the subject an effective amount of an opioid and a dopamine type 3 receptor (D3R) agonist and/or an effective amount of an opioid and a dopamine type 1 receptor (D1R) antagonist.

D3R agonists can be any molecule that activates the D3R. In some embodiments, the agonist may specifically activate D3R. In some embodiments, the agonist may predominantly activate D3R over other dopamine receptors. Nonlimiting examples of a dopamine 3 receptor agonist of the inventive concept include enafadotride, cabergoline, PD 128907, pramipexole (Mirapex), pergolide, and rotigotine (NeuproTM), singly or in any combination and/or ratio.

In some embodiments, subjects to whom the methods of the inventive concept are applied can receive a lower dose of an opioid when it is administered in combination with a D3R agonist of the inventive concept. By “lower dose” is meant a reduced amount of an opioid relative to the amount that the subject is, may be taking or has been taking, without a D3R agonist in combination, e.g., at least about 5%, 10%, 15%, 20%, 25%, 30%, 35%, 40%, 45%, or 50% less. In some embodiments, this lower dose may be about 5%, 10%, 15%, 20%, 25%, 30%, 35%, 40%, 45%, or 50%, less than the maximum dose of an opioid that may be administered, and/or what is considered by one of skill in the art to be the maximum safe dose of an opioid that may be administered to the subject. Nevertheless, in some embodiments, for example, in, but not limited to, nerve healing, a larger, such as at or nearer, e.g., at least about 50%, 55%, 60%, 65%, 70%, 75%, 80%, 90%, 95%, or 100% of the maximum, one time dose of the opioid may be administered without departing from the methods of the inventive concept.

In further embodiments of the inventive concept, a dopamine 1 receptor (D1R) antagonist can be administered in the methods described herein and/or included in the compositions described herein.

In some embodiments, the methods of the inventive concept are carried out without the inclusion of a D1R antagonist and in some embodiments, the compositions do not comprise or include a D1R antagonist. For example, in some embodiments, a composition of the inventive concept can comprise (a) at least one opioid; (b) at least one dopamine type 3 receptor agonist; and (c) a pharmaceutically acceptable carrier, excipient or diluent, with the proviso that the composition does not comprise a D1R antagonist. As another example, in some embodiments, the present inventive concept provides a method of treating fibrosis across a wide range of disease and injury states, and aging in a subject in need thereof, comprising administering to the subject an effective amount of an opioid and a dopamine type 3 receptor (D3R) agonist, with the proviso that a D1R antagonist is not administered to the subject.

Nonlimiting examples of a D1R antagonist include ecopipam (SCH 39166), SCH 23390, SKF 83566, singly or in any combination and/or ratio.

It is contemplated that any specific opioid, D3R agonist and/or D1R antagonist, singly or in any combination, can be excluded from the methods and/or compositions of the inventive concept.

In further embodiments, the present inventive concept additionally provides compositions that can be employed in the methods of the inventive concept. Thus, in one embodiment, the present inventive concept provides a composition comprising: (a) at least one opioid; (b) at least one D3 receptor agonist; and (c) a pharmaceutically acceptable carrier, excipient or diluent. The composition can be a pharmaceutical composition or formulation that can comprise additional therapeutic agents or techniques. See, e.g., Remington, The Science And Practice of Pharmacy (latest edition).

Nonlimiting examples of an additional agent that can be included in a pharmaceutical composition or formulation and/or can be administered according to the methods of the inventive concept include, e.g., analgesic agents, non-steroid anti-inflammatory (NSAID) agents (e.g., Ibuprofen, Naproxen, Ketoprofen, Diclofenac, Fenoprofen, Ketoroloac, Meloxicam, Indomethacin, Piroxicam, Cox-2 inhibitors, etc.), salicylates (e.g., aspirin, magnesium salicylate, diflunisal, etc.), acetaminophen, codeine, chlorpheniramine, pseudoephedrine, homatropine, triptans, and/or ergots, individually, or in any combination, and/or in any ratio.

Additional agents that can be included in a pharmaceutical composition or formulation and/or can be administered according to the methods of the inventive concept include, e.g., antioxidants such as vitamins C and E, selenium, flavonoids, tannins, phenols, lignans, and carotenoids, such as beta-carotene, lycopene, lutein, and zeaxanthin, and anti-aging drugs candidates such as metformin, rapamycin, resveratrol and NAD+ precursors, individually, or in any combination, and/or in any ratio as might be appreciated by one of skill in the art.

In terms of administration of a composition of the inventive concept, the most suitable route in any given case will depend on the nature and severity of the condition and/or the pharmaceutical formulation being administered. The active agents described herein can be formulated for administration in a pharmaceutical carrier in accordance with known practices.

The compositions of the present invention may be suitable for and formulated for parenteral, oral, inhalation spray, topical (i.e., both skin and mucosal surfaces, including airway surfaces), rectal, nasal, buccal (e.g., sub-lingual), vaginal or implanted reservoir administration, etc. where the most suitable route in any given case will depend on the nature and severity of the condition being treated in combination with the drug profile of the compound described herein as would be understood by one of ordinary skill in the art.

For topical administration, suitable forms include, but are not limited to an ointment, cream, emulsion, microemulsion, a gel, a dispersion, a suspension, a foam, an aerosol, a liquid, a droplet, and suitable transdermal delivery systems known in the art, such as patches and bandages, dressing, gauze and the like including the medicament described herein. Topical administration may further include articles of clothing such as socks or hosiery including the medicament described herein.

The term “parenteral” as used herein includes subcutaneous, intradermal, intravenous, intramuscular, intraperitoneal, intra-articular, intra-synovial, intrasternal, intrathecal, intrahepatic, intralesional, and/or intracranial, injection or infusion techniques. It will be appreciated that injection encompasses techniques involving, e.g., a volume, typically a smaller volume, of a medication delivered in a shot or bolus over a short period of time, such as administered via syringe with the application of pressure. On the other hand, infusion encompasses techniques involving, e.g., a volume, frequently a larger volume, of medication delivered over a longer period of time, such as more than about 10 minutes, more than about 15 minutes, more than about 30 minutes, or over an even longer (hours, days, weeks) period of time.

Compositions for injection will include the active ingredient together with suitable carriers including propylene glycol-alcohol-water, isotonic water, sterile water for injection (USP), emulPhor™-alcohol-water, cremophor-EL™, polyvinyl pyrrolidone, lecithin, arachis oil or sesame oil, with other additives for aiding solubility or preservation may also be included, or other suitable carriers known to those skilled in the art. Accordingly, these carriers may be used alone or in combination with other conventional solubilizing agents such as ethanol, propylene glycol, or other agents known to those skilled in the art.

Compositions for oral administration may be, for example, solid preparations such as tablets, sugar-coated tablets, hard capsules, soft capsules, granules, powders, gelatins, and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like.

Where the compounds described herein are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges, gelatins, and pastilles, wherein the active ingredient is formulated with a carrier such as sugar and acacia, tragacanth or gelatin and glycerin.

In particular embodiments, the present invention provides a pharmaceutical formulation including the compound described herein wherein the pharmaceutical formulation is a parenteral formulation. In some embodiments, the parenteral formulation is an intravenous formulation. In some embodiments the parenteral formulation is an intraperitoneal formulation. In other embodiments, the present invention provides a pharmaceutical formulation including the compound described herein wherein the pharmaceutical formulation is an oral formulation.

According to the present invention, methods of the inventive concept include administering an effective amount of a composition of the present invention as described above to the subject. The effective amount of the composition, the use of which is in the scope of present invention, will vary somewhat from subject to subject, and will depend upon factors such as the age and condition of the subject and the route of delivery. Such dosages can be determined in accordance with routine pharmacological procedures known to those skilled in the art. A composition of the present invention can comprise the active agents in an amount ranging from a lower limit from about 0.01, 0.05, 0.10, 0.50, 1.0, 5.0, or 10% to an upper limit ranging from about 10, 20, 30, 40, 50, 60, 70, 80, 90, 95, 96, 97, 98, 99, or 100% by weight of the composition. In some embodiments, the active agents include from about 0.05 to about 95% by weight of the composition. In other embodiments, the active agents can include from about 0.05 to about 60% by weight of the composition. In still other embodiments, the active agents include from about 0.05 to about 10% by weight of the composition.

Embodiments of the present invention further provide kits comprising, consisting essentially of or consisting of one or more containers having pharmaceutical dosage units comprising an effective amount of at least one D1 receptor antagonist, at least one D3 receptor agonist and optionally an opioid agonist, wherein the container is packaged with optional instructions for the uses described herein.

The effective dosage of any specific active agent will vary somewhat from composition to composition, patient to patient, and will depend upon the condition of the patient and the route of delivery. As a general proposition, a dosage from about 0.1 to about 50 mg/kg will have therapeutic efficacy, with still higher dosages potentially being employed for oral administration, wherein aerosol administration is usually lower than oral or intravenous administration. Toxicity concerns at the higher level may restrict intravenous dosages to a lower level such as up to about 10 mg/kg, all weights being calculated based upon the weight of the active base, including the cases where a salt is employed. For the opioid component, a typical dose may range from about 0.1 mg/day to about 6 mg/day for intravenous or intramuscular administration. A dosage from about 0.1 mg/day to about 60 mg/day may be employed for oral administration. The D3R agonist dose may range from about 0.05 mg/day to about 50 mg/day.

In particular embodiments, administration to a subject such as a human, a dosage of from about 0.1 mg/day (e.g., about 1.0 mg/day), up to about 60 mg/day for opioid and from about 0.05 mg/day to about 100 mg/day (e.g., about 50 mg/day) for D3R agonist or more can be employed. Depending on the solubility of the particular formulation of active agents administered, the dose, which in some embodiments, can be in hourly (e.g., every four hours; every six hours; every 12 hours, etc.), daily (e.g., once a day; twice a day, etc.), weekly (e.g., once a week; twice a week; four times a week, etc.), monthly and/or yearly increments, can be divided among one or several unit dose administrations.

### Definitions

As used herein, “a” or “an” or “the” can mean one or more than one. Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of the inventive concept, dose, time, temperature, and the like, is meant to encompass variations of 20%, 10%, 5%, 1%, 0.5%, or even 0.1% of the specified amount.

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of the inventive concept is not intended to be interpreted to be equivalent to “comprising.”

Nonetheless, in some embodiments, “comprise” may also encompass, and, in some embodiments, may refer to the expressions “consist essentially of” and/or “consist of.” Thus, the expression “comprise” can also refer to and encompass embodiments, wherein that which is claimed “comprises” specifically listed elements does not include further elements, as well as embodiments wherein that which is claimed “comprises” specifically listed elements may and/or does encompass further elements, or encompass further elements that do not materially affect the basic and novel characteristic(s) of that which is claimed. For example, that which is claimed, such as a method, kit, system, etc. “comprising” specifically listed elements also encompasses, for example, a method, kit, system, etc. “consisting of,” i.e., wherein that which is claimed does not include further elements, and, for example, a method, kit, system, etc. “consisting essentially of,” i.e., wherein that which is claimed may include further elements that do not materially affect the basic and novel characteristic(s) of that which is claimed.

The term “modulate,” “modulates” or “modulation” refers to enhancement (e.g., an increase) or inhibition (e.g., a reduction) in the specified activity.

The term “management” as used herein refers to the ability to affect a method, process, state of being, disorder or the like. The effect may be that of prevention, treatment or modulation.

By the terms “treat,” “treating” or “treatment of,” it is intended that the severity of the disorder or the symptoms of the disorder are reduced, or the disorder is partially or entirely eliminated, as compared to that which would occur in the absence of treatment. Treatment does not require the achievement of a complete cure of the disorder.

By the terms “preventing” or “prevention,” it is intended that the inventive methods eliminate or reduce the incidence or onset of the disorder, as compared to that which would occur in the absence of the measure taken. Alternatively stated, the present methods slow, delay, control, or decrease the likelihood or probability of the disorder in the subject, as compared to that which would occur in the absence of the measure taken.

By the terms “functional recovery” and improving functional recovery,” it is intended that the inventive methods improve recovery of normal functions in tissues/cells, for example, nervous tissue/nerve cells, that may be lost as a result of a disorder and/or injury, such as in a neurodegenerative disorder and/or a nerve injury. Functional recovery is intended to include partial recovery of normal functions, as well as near complete and/or complete recovery/restoration of normal function in cells/tissues. Moreover, functional recovery may not occur immediately following treatment with/administration of compositions according to methods of the present inventive concept, and/or may require more than one treatment with/administration of compositions according to methods of the present inventive concept. Treatment/administration to achieve functional recovery may include, in some embodiments, treatment/administration over a period of time, for example, over minutes, hours, days, weeks and/or months, and/or may include multiple, i.e., more than one, treatments/administrations of compositions by methods according to the present inventive concept as may be envisioned by one of skill in the art without limitation.

A “therapeutically effective” or “effective” amount is intended to designate a dose that causes a relief of symptoms of a disease or disorder as noted through clinical testing and evaluation, patient observation, and/or the like. “Effective amount” or “effective” can further designate a dose that causes a detectable change in biological or chemical activity. The detectable changes may be detected and/or further quantified by one skilled in the art for the relevant mechanism or process. Moreover, “effective amount” or “effective” can designate an amount that maintains a desired physiological state, i.e., reduces or prevents significant decline and/or promotes improvement in the condition of interest. As is generally understood in the art, the dosage will vary depending on the administration routes, symptoms and body weight of the patient but also depending upon the compound being administered.

“Tolerance” refers to a declining response to treatment over time.

“Drug tolerance” means a decreasing response to repeated constant doses of a drug or the need for increasing doses to maintain a constant response.

D3R is dopamine 3 receptor agonist.

PRAM is pramipexole, a clinically available dopamine 3 receptor agonist with preference for dopamine 3 receptors.

Morph is morphine, which is an opioid often prescribed for pain.

Oxycodone is a semi-synthetic opioid often prescribed for pain.

“In combination with” means sufficiently close in time to produce a combined effect (that is, in combination with can be simultaneously, or it can be two or more events occurring within a short time period before or after each other). In some embodiments, the administration of two or more compounds in combination with means that the two compounds are administered closely enough in time that the presence of one alters the biological effects of the other. The two compounds can be administered in the same or different formulations or sequentially. Such concurrent administration can be carried out by mixing the compounds prior to administration, or by administering the compounds in two different formulations, for example, at the same point in time but at different anatomic sites or using different routes of administration.

A “subject” as used herein can be a human subject and can include, but is not limited to a patient. The subject may be male or female and may be of any race or ethnicity, including, but not limited to, Caucasian, African-American, African, Asian, Hispanic, Indian, etc. The subject may be of any age, including newborn, neonate, infant, child, juvenile, adolescent, adult, and geriatric. In some embodiments, the subject is over 30, 40, 50, 60, 70, 80 or 90 years of age. A subject can also include an animal subject, including mammalian subjects such as canines, felines, bovines, caprines, equines, ovines, porcines, rodents (e.g., rats and mice), lagomorphs, primates (including non-human primates), etc., avian subjects (e.g., fowl, parrots, raptors etc.), and the like, for prevention and treatment purposes as well as veterinary medicine and/or pharmaceutical drug development purposes. A subject of the inventive concept can be experiencing fibrosis across a wide range of disease and injury states as well as age-related events.

### Example 1: Adjuvant Application of a Dopamine D3 Receptor Agonist to an Ineffective Morphine Dose Provides Analgesia in a Model of Chronic Pain

The recording of compound action potentials (CAPs) from peripheral nerves is a routine clinical investigative procedure in the operating room. Parameters such as amplitude, area, conduction velocity, and latency are used as measures for the functional integrity of the nerve. Pathology produces reduction in conduction velocities, desynchronization of compound nerve action potentials and, in severe disease, complete conduction block, leading to observable changes in these measurements. “A Comparative Study of Compound Action Potentials and Currents.”-K. W. Kim, D. J. Engle & R. J. Sclabassi

The CAP is an indicator of electrical activity in the nerve that is required for normal transmission of sensory information. After nerve injury, this electrical activity is lost. This correlates with the development of pain on the injured side. In animals treated chronically with the morphine+PPX combination, As shown below, normal pain thresholds are maintained after injury AND that some degree of measurable electrical activity is present in the injured nerve. So there is an association between those two events. This preservation or recovery of function is believed to be related to the ability of the drug to reduce fibrosis and/or inflammation in the nerve after injury, allowing the nerve to conduct electrical impulses.

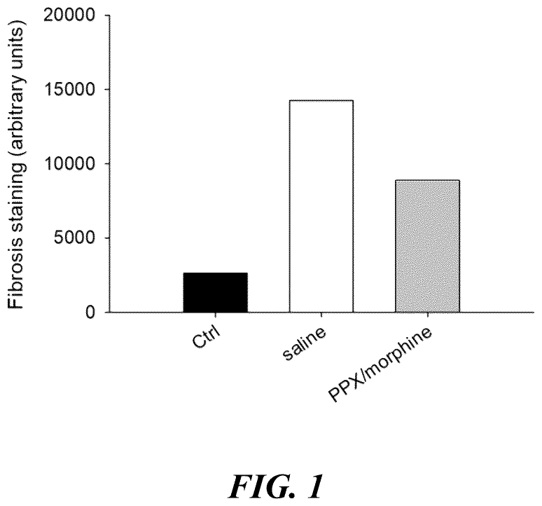

The effect on fibrosis resulting from sciatic nerve injury was examined in vitro, and results of long-term treatment are shown in FIG. 1. Under control conditions, the sciatic nerve expresses only low levels of fibrosis (black bar). After injury, and after long-term treatment with saline only, the fibrosis level around the site of the injury is significantly increased (white bar). However, long-term treatment with a pramipexole (PPX) and morphine combination (0.5 mg/kg and 1 mg/kg each) exhibits a significant reduction in the fibrosis signal (gray bar).

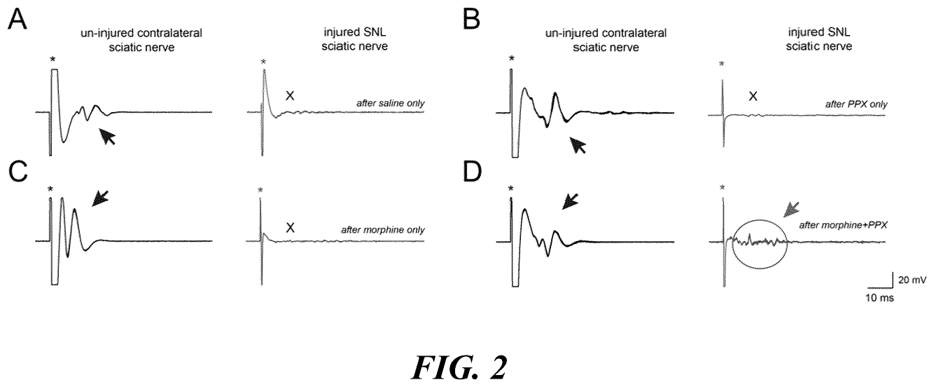

The effects in vitro of long-term treatments on sciatic nerve function were assessed by the response profiles of these nerves to electrical stimulation as shown in FIG. 2. Panel A shows on the left the compound action potentials (CAPs, s. arrow) of a representative animal that had undergone sciatic nerve injury and was exposed to daily treatments of 100 μl saline (the drug carrier for the experiments in B-D). In the same animal, on the injured side, stimulation of the nerve failed to elicit a similarly strong CAP as on the injured contralateral side (labeled by ‘X’). Panel B shows on the left the CAPs of a representative animal that had undergone sciatic nerve injury and was exposed to daily treatments of 100 μl PPX (0.5 mg/kg, dissolved in saline). In the same animal, on the injured side, stimulation of the nerve failed to elicit a similarly strong CAP as on the injured contralateral side (labeled by ‘X’). Panel C shows on the left the CAPs of a representative animal that had undergone sciatic nerve injury and was exposed to daily treatments of 100 μl morphine (1 mg/kg, dissolved in saline). In the same animal, on the injured side, stimulation of the nerve failed to elicit a similarly strong CAP as on the injured contralateral side (labeled by ‘X’). Panel D shows on the left the CAPs of a representative animal that had undergone sciatic nerve injury and was exposed to daily treatments of 100 μl PPX: morphine (0.5 mg/kg and 1 mg/kg, respectively, dissolved in saline). In the same animal, on the injured side, stimulation of the nerve was able to produce a CAP, albeit less strong than on the injured contralateral side (s. arrow).

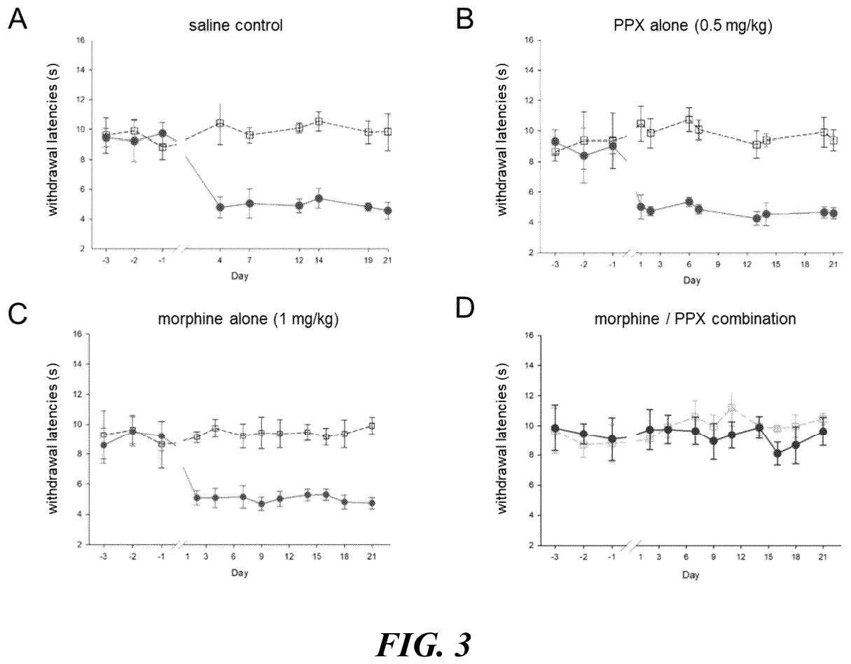

The effects of different long-term treatments on pain withdrawal reflex latencies in vivo are shown in FIG. 3. Panel A shows the effects of saline treatment on reflex latencies after unilateral sciatic nerve injury. Hollow squares show the reflex data of the contralateral (un-injured) side of the animal, which stay stable over time. Filled circles show the reflex data of the injured side of the animal, and they show that saline treatment did not lead to a recovery of withdrawal latencies to pre-injury data (days−3 to −1). Panel B shows the effects of PPX treatment (0.5 mg/kg) on reflex latencies after unilateral sciatic nerve injury. Hollow squares show the reflex data of the contralateral (un-injured) side of the animal, which stay stable over time. Filled circles show the reflex data of the injured side of the animal. As with saline treatment, treatment with PPX alone did not lead to a recovery of withdrawal latencies to pre-injury data (days −3 to −1). Panel C shows the effects of morphine treatment (1 mg/kg) on reflex latencies after unilateral sciatic nerve injury. Hollow squares show the reflex data of the contralateral (un-injured) side of the animal, which stay stable over time. Filled circles show the reflex data of the injured side of the animal. As with saline or PPX treatment, treatment with morphine alone did not lead to a recovery of withdrawal latencies to pre-injury data (days −3 to −1). Panel D shows the effects of the PPX: morphine treatment (0.5 mg/kg and 1 mg/kg, respectively) on reflex latencies after unilateral sciatic nerve injury. Hollow squares show the reflex data of the contralateral (un-injured) side of the animal, which stay stable over time. Filled circles show the reflex data of the injured side of the animal. Unlike PPX or morphine treatment alone, treatment with the PPX: morphine combination maintained withdrawal latencies of the injured side at the levels of the pre-injury data (days −3 to −1).

These data indicate that the beneficial effects of the combination treatment may indicate a novel treatment approach to treat chronic and opioid-resistant pain, and that it is independent of the type of chronic pain, be it centrally or peripherally-induced.

In addition, the electrophysiological data indicate that the drug combination may also have a restorative or protective effect on nerve function in the injured nerves.

### Example 2: Adjuvant Application of a Dopamine D3 Receptor Agonist and an Opioid for Treating Inflammation Associated With Neurodegenerative Diseases

Neurodegenerative diseases (e.g., Alzheimer's disease [AD], Parkinson's disease [PD], Multiple Sclerosis [MS], and Amyotrophic lateral sclerosis [ALS]) affect over 6.5 M Americans today, and the numbers are expected to double in the next 30 years. Despite a robust commitment by the pharmaceutical industry to develop solutions to slow down or halt the progress of the pathologies, there are no clinical outcomes that have significantly modified disease progression or improved patient's quality of life [2, 3]. Even the most recent breakthrough, using an antibody approach, was only successful in mild-to-moderate AD patients and not tested for more than 18 months (https://www.bbc.com/news/health-63749586). Thus, there is an urgent need to identify new approaches that will lead to more substantial benefits than those currently used in the clinic.

A hallmark of neurodegenerative diseases is the increase of inflammatory markers in the affected tissues, often associated with fibrosis (AD: [4]; MS: [5], ALS: [6]), and AD, MS, and ALS share many of the same principal mediators and extracellular components [7, 8]. This commonality across the different disease types presents a unique opportunity for novel treatment options that in turn reduce the disease symptoms of not only AD, but across the spectrum of neurodegenerative diseases.

Experimental Design: Surgery: Male mice (C57BL/6, ˜8 weeks of age upon arrival) are used in this study. Following anesthesia, body hair is removed from the left hind leg of the animals with Nair™ lotion and wiped off. An alcohol wipe is then used to remove any excess hair from the leg. The shaved area is treated with 3 alternate applications of betadine and 70% ethanol. Animals are laid on their chest, and the left hind leg is extended and held in a position with the femur 90° to the spine. An incision is made in the skin parallel, 3-4 mm below the femur and the skin is freed from the muscle by puncturing the connective tissue. Blunt forceps are used to puncture the connective tissue between the gluteus superficialis and the biceps femoris muscles. Blunt forceps are used to carefully widen the gap between the two muscles, exposing the sciatic nerve. Blunt tip forceps are used to free approximately 10 mm of the sciatic nerve from the surrounding connective tissue. With use of a microscope, 3 ligatures are made with a double knot, about 2-3 mm apart, proximal to the trifurcation of the sciatic nerve. These ligatures are made with 4-0 braided silk suture. Constriction of the nerve is minimal to prevent arresting epineural blood flow. Sutures are used to close the muscle layer and skin.

Behavioral testing: Hargreaves' testing is used to determine thermal pain withdrawal latencies. Prior to each test, animals are acclimatized on the Hargreaves platform and Plexiglas enclosure for 1 hour, and baseline withdrawal latencies are determined for each animal, with five independent trials performed per hind paw per session and a mean latency calculated for each day. These pre-surgery tests are performed in an alternating way on both hindlimbs and are administered 3 days/week, starting one week prior to surgery. Following surgery, animals are tested 3×/week for up to 8 weeks. Four cohorts of animals are used (each N=8): Cohort 1 receive daily sham injections (i.p.; 100 μl; 0.9% sterile NaCl) between 8-11 AM in the morning, to avoid possible conflicts with the circadian cycle, and will serve as the injury control to the drug paradigms in cohorts 2-4; Cohort 2 receive from the day after the SNL injections of 1 mg/kg morphine and 0.5 mg/kg pramipexole; Cohort 3 receive from two weeks after SNL injections of 1 mg/kg morphine and 0.5 mg/kg pramipexole. These two cohorts will test if the recovery of function can be achieved directly after injury or even after a prolonged time under chronic pain. Cohort 4 receive injections of 1 mg/kg morphine and 0.5 mg/kg pramipexole from 3 days prior to SNL. This cohort will address the question if the drug combination can be used as a preventative measure. All Injections are given daily, for a duration of up to 4 weeks post-SNL. Throughout the treatment paradigm, thermal pain reflex latencies are measured from each cohort 3×/week on the Hargreaves platform, starting ˜1 hr after drug injection. These studies are approved under the ECU animal user protocol Q273d.

Tissue collection: Following the behavioral tests, animals are humanely euthanized, and sciatic nerves and spinal cords are harvested to analyze functional changes related to the nerve injury and the drug treatments. The amount of the scar around the nerve is assessed, similar to previous work [9], and the treatment-induced changes on the extent of the fibrotic tissues is quantified.

### References

1. Association, A., 2022 Alzheimer's Disease Facts and Figures, A. s. Association, Editor. 2022.
2. van Bokhoven, P., et al., The Alzheimer's disease drug development landscape. Alzheimers Res Ther, 2021. 13(1): p. 186.
3. Cummings, J., et al., Alzheimer's disease drug development pipeline: 2022. Alzheimers Dement (N Y), 2022. 8(1): p. e12295.
4. Nicoll, J. A., et al., Cerebral amyloid angiopathy plays a direct role in the pathogenesis of Alzheimer's disease. Pro-CAA position statement. Neurobiol Aging, 2004. 25(5): p. 589-97; discussion 603-4.
5. van Horssen, J., C. D. Dijkstra, and H. E. de Vries, The extracellular matrix in multiple sclerosis pathology. J Neurochem, 2007. 103(4): p. 1293-301.
6. Peters, S., et al., The TGF-beta System As a Potential Pathogenic Player in Disease Modulation of Amyotrophic Lateral Sclerosis. Front Neurol, 2017. 8: p. 669.
7. D'Ambrosi, N. and S. Apolloni, Fibrotic Scar in Neurodegenerative Diseases. Front Immunol, 2020. 11: p. 1394.
8. Heindryckx, F. and J. P. Li, Role of proteoglycans in neuro-inflammation and central nervous system fibrosis. Matrix Biol, 2018. 68-69: p. 589-601.
9. Johnson, T. L., et al., The dopamine D3 receptor knockout mouse mimics aging-related changes in autonomic function and cardiac fibrosis. PLoS One, 2013. 8(8): p. e74116.

The foregoing is illustrative of the present inventive concept, and is not to be construed as limiting thereof. The invention is defined by the following claims, with equivalents of the claims to be included therein. All publications, patent applications, patents, patent publications, and any other references cited herein are incorporated by reference in their entireties for the teachings relevant to the sentence and/or paragraph in which the reference is presented.

That which is claimed:

1. A method of reducing, treating, and/or managing fibrosis associated with disease or aging, reducing or inhibiting disease progression and inflammation, and/or for improving functional recovery comprising administering to the subject an effective amount of an opioid and a dopamine type 3 receptor (D3R) agonist, or an effective amount of an opioid and a dopamine type 1 receptor (D1R) antagonist.

2. The method of claim 1, wherein the method comprises administering an opioid and a dopamine type 3 receptor (D3R) agonist.

3. The method of claim 1, wherein the method comprises administering an opioid and a dopamine type 1 receptor (D1R) antagonist.

4. The method of any one of claims 1-3, wherein the opioid, and the D3R agonist or the D1R antagonist are administered in the same composition.

5. The method of any one of claims 1-3, wherein the opioid, and the D3R agonist or the D1R antagonist are administered in separate compositions.

6. The method of claim 5, wherein the opioid, and the D3R agonist or the D1R antagonist are administered by the same route.

7. The method of claim 5, wherein the opioid, and the D3R agonist or the D1R antagonist are administered by different routes.

8. The method of any one of claims 1-7, wherein the opioid, and the D3R agonist or the D1R antagonist are administered via one or more of oral administration, injection, infusion, patch, and a surgically implanted pump.

9. The method of any one of claims 1-8, further comprising delivering to the subject an additional therapeutic agent.

10. The method of any one of claims 1-9, wherein the subject is a human.

11. A composition comprising:
(a) at least one opioid;
(b) at least one dopamine type 3 receptor agonist or a dopamine type 1 receptor antagonist;
(c) an additional therapeutic agent; and
(d) a pharmaceutically acceptable carrier, excipient or diluent.

12. A kit for reducing, treating and/or managing fibrosis associated with disease or aging, reducing or inhibiting disease progression and inflammation, and for improving functional recovery comprising at least one opioid and at least one dopamine type 3 receptor agonist or a dopamine type 1 receptor (D1R) antagonist.

13. A method of reducing, treating and/or managing a neurodegenerative disease in a subject, the method comprising:
taking a baseline compound action potential (CAP) measurement;
treating the subject with at least one opioid and at least one dopamine type 3 receptor (D3R) agonist or a dopamine type 1 receptor (D1R) antagonist;
taking an additional baseline compound action potential (CAP) measurement after treating the subject with the at least one opioid and at least one D3R agonist or the D1R antagonist; and
assessing whether the CAP (amplitude, area, conduction velocity, and latency) is stabilized or improves over time.

14. The method of claim 13, wherein treating and/or managing the neurodegenerative disease comprises treating and/or managing inflammation associated with the neurodegenerative disease.

15. A method of reducing, treating, and/or managing inflammation resulting from injury, disease, and/or aging in a subject, the method comprising administering to the subject an effective amount of an opioid and a dopamine type 3 receptor (D3R) agonist, or an effective amount of an opioid and a dopamine type 1 receptor (D1R) antagonist.

16. The method of claim 15, wherein reducing, treating, and/or managing of inflammation comprises reducing, treating, and/or managing of fibrosis associated with the inflammation.

17. The method of claim 15 or 16, wherein the method comprises administering an opioid and a dopamine type 3 receptor (D3R) agonist.

18. The method of claim 15 or 16, wherein the method comprises administering an opioid and a dopamine type 1 receptor (D1R) antagonist.

19. The method of any one of claims 15-18, wherein the opioid, and the D3R agonist or the D1R antagonist are administered in the same composition.

20. The method of any one of claims 15-18, wherein the opioid, and the D3R agonist or the D1R antagonist are administered in separate compositions.

21. The method of claim 20, wherein the opioid, and the D3R agonist or the D1R antagonist are administered by the same route.

22. The method of claim 20, wherein the opioid, and the D3R agonist or the D1R antagonist are administered by different routes.

23. The method of any one of claims 15-22, wherein the opioid, and the D3R agonist or the D1R antagonist are administered via one or more of oral administration, injection, patch, and a surgically implanted pump.

24. The method of any one of claims 15-23, further comprising delivering to the subject at least an additional therapeutic agent.

25. The method of any one of claims 15-24, wherein the subject is a human.

26. A kit for reducing, treating and/or managing inflammation associated with disease or aging, reducing or inhibiting disease progression and/or fibrosis associated with the inflammation, and for improving functional recovery comprising at least one opioid and at least one dopamine type 3 receptor agonist or a dopamine type 1 receptor (D1R) antagonist.

27. The composition of claim 11 for use in reducing, treating, and/or managing inflammation resulting from injury, disease, and/or aging in a subject.

28. The composition of claim 27, wherein reducing, treating, and/or managing of inflammation comprises reducing, treating, and/or managing of fibrosis associated with the inflammation.

29. Use of an effective amount of an opioid and a dopamine type 3 receptor (D3R) agonist, or an effective amount of an opioid and a dopamine type 1 receptor (D1R) antagonist in the manufacture of a medicament for reducing, treating, and/or managing inflammation resulting from injury, disease, and/or aging in a subject.

30. The use of claim 29, wherein reducing, treating, and/or managing of inflammation comprises reducing, treating, and/or managing of fibrosis associated with the inflammation.
